(19) United States
(12) Patent Application Publication
(10) Pub. No.: US 2006/0263879 A1
(43) Pub. Date: Nov. 23, 2006

(54) Methods and systems relating to embryonic stem cell lines

(75) Inventors: Carlos Simon Valles, Godella (ES); Ana Krtolica, San Francisco, CA (US); Russell A. Foulk, Reno, NV (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: StemLifeLine, Inc., San Carlos, CA (US)

(21) Appl. No.: 11/317,887

(22) Filed: Dec. 22, 2005

Related U.S. Application Data
(60) Provisional application No. 60/640,637, filed on Dec. 30, 2004.
(60) Provisional application No. 60/674,472, filed on Apr. 25, 2005.

(51) Int. Cl. C12N 5/08

(52) U.S. Cl. 435/366

(57) ABSTRACT

The invention provides methods for generating customized hESC and lines thereof for future use in the treatment of genetic relations. In one aspect, the customized hESC lines are grown using feeder cells from biologically or genetic relations in order to avoid contamination by foreign pathogens or exposure to foreign antigens.

### RELATED APPLICATIONS

This application claims priority to U.S. provisional applications having Ser. Nos. 60/640,637 and 60/674,472, filed on Dec. 30, 2004 and Apr. 25, 2005, respectively, and entitled “METHODS AND SYSTEMS RELATING TO EMBRYONIC STEM CELL LINES”, the entire contents of both of which are incorporated by reference herein.

### FIELD OF THE INVENTION

The invention relates to the generation of embryonic stem cell lines under particular culture conditions. The invention also relates to the future use of stem cell lines in genetic relations.

### BACKGROUND OF THE INVENTION

Generation and use of murine embryonic stem cells (ESC) is now routine in the art. Generation of human embryonic stem cells (hESC) is a more recent development. Thomson described the ability to generate primate ESC from embryos. (See U.S. Pat. No. 6,200,806 B1, for example.) The ability to generate and maintain hESC has relied predominately on co-culture with feeder cells derived from non-human species, including mouse feeder cells. However, this co-culture system risks contamination of the hESC with pathogens and antigens specific to mice and never before seen in humans. Additionally, there is a risk of xenotransplantation of mouse cells into human subjects due to this co-culture system.

Accordingly, other researchers have begun to study the feasibility of human feeder cells in the generation and maintenance of hESC. Various human feeder cells have been disclosed for maintaining hESC in an undifferentiated state including adult marrow cells (Freed, 2002, PNAS USA 99:1755-1757), fetal muscle (Bjorklund et al., 2002, PNAS USA 99:2344-2349), fetal skin (Bjorklund et al., 2002, PNAS USA 99:2344-2349), adult fallopian tube (Bjorklund et al., 2002, PNAS USA 99:2344-2349), foreskin (U.S. Pat. No. 6,534,052; Kehat and Gepstein, 2003, Heart Fail Rev 8:229-236), tissue-derived stromal cells (Nir et al., 2003, Cardiovasc Res 58:313-323), embryonic fibroblasts (Kehat and Gepstein, 2003, Heart Fail Rev 8:229-236), and placental fibroblasts. However these latter approaches are still susceptible to contamination issues and any ESC generated on such third party feeder cells will need to be screened for pathogen content before any in vivo use. There exists a need for hESC generation methods that substantially reduce and preferably eliminate contamination concerns. There also exists a need for methods that provide hESC tailored to each person desiring such cells. There further exists a need for methods, systems and processes for storing and making such customized hESC available to future users.

### SUMMARY OF THE INVENTION

The invention relates in part to the generation and maintenance of human embryonic stem cell (hESC) lines for use in particular biologically, and preferably genetically, related family members. The invention provides inter alia services to those interested in establishing and storing one or more hESC lines for future use by themselves or certain of their family members. The services allow for storage, tracking and retrieval of the samples as well as the use of the hESC lines either in vitro or in vivo. The invention further provides systems and processes for accomplishing such services. Also included in the invention is the generation of a hESC line bank that houses samples of hESC lines (and optionally feeder cell and/or serum samples also), optionally in conjunction with a database having information records, including identification records, for each sample.

In one aspect, the invention provides a method for establishment and use of a hESC cell line comprising generating a hESC line, for use in treating a familial subject, optionally in the future. The familial subject may be a genetic parent or a genetic sibling, or it may be isogenic with the hESC line. The method comprises generation and storage, and optionally retrieval and transfer of the hESC line. In other instances, destruction of the sample may also be provided.

In one embodiment, the method comprises generating a hESC line, and storing the hESC line in connection with a database for directing use of the hESC line in treating a familial subject. In one embodiment, the database is a computer database. The database comprises one or more information records. Each information record may include one or more fields, and each field may include one or more subfields. One field or subfield may be an identification record, which optionally at least comprises a code that identifies the sample but does not reveal confidential information about the sample, such as donor name and the like.

The information record may comprise, in fields or subfields, information relating to identification and location of the stored hESC line (including an identification code), identification and location of a stored feeder and/or serum sample (if any), date of storage of the sample, date of hESC line generation, characteristics of the stored hESC line (including but not limited to differentiative capacity, phenotype of the stored line according to for example stem cell markers, proliferation rate and/or doubling time of stored line, hESC line colony morphology including if available electronic images thereof), donor information (including the name of the egg donor, name of the sperm donor, relations of the donors including children and siblings, pathogen testing of the donors, pathogen testing of the stored samples, and the like. Information may be organized into fields such as but not limited to a stem cell line identification field, a stem cell line generation protocol field, a stem cell line profile field, a feeder cell sample field or a serum sample field, and like.

In one embodiment, the method further comprises storing the human feeder cells in the bank and listing the human feeder cells in the information record, wherein the human feeder cells are derived from a biological or genetic relation of the human embryonic stem cell line and were used to generate the human embryonic stem cell line. In another embodiment, the method further comprises storing the human serum in the bank and including the human serum in the information record, wherein the human serum is derived from a biological or genetic relation of the human embryonic stem cell line and was used to generate the human embryonic stem cell line. The feeder cells may be those described below.

The method may further provide screening the sample for pathogen content prior to storage and/or transfer. The hESC line may be further analyzed (e.g., haplotyped, karyotyped, genotyped, etc.) prior to storage and/or transfer.

The method may further comprise retrieving the stored samples, and optionally transferring the samples to a client (e.g., an owner of the line) or a third party. If the samples are cryopreserved, the method may further comprise thawing the samples. In still another embodiment, the method further comprises culturing the samples.

The method may further comprise levying a charge for each stored sample based on length of storage, and optionally number of stored samples.

In another aspect, the invention provides a computer readable medium or combination of computer-readable media, containing a program for maintaining a computer-based hESC line database or registry, the program comprising code to effect storing information about a human embryonic stem cell line, optionally in the form of one or more information records, and thereby creating an electronic database. In one embodiment, the information record comprises information corresponding to the identity and storage location of the human embryonic stem cell line. In other embodiments, the information is organized as fields and/or subfields and relates to the characteristics of the line, the feeder cells and/or the serum including but not limited to the differentiative capacity of the human embryonic stem cell line, the growth characteristics of the human embryonic stem cell line, and the like.

In yet another aspect, the invention provides a method for providing embryonic stem cell line information to a client, the method comprising computer-implemented steps of storing, searching and selecting information about a human embryonic stem cell line which may optionally be organized in information records in a database based on particular criteria. Such criteria include an identification code, the location of the stored samples, the number of stored samples, the disposition and/or status of such stored samples, the differentiative capacity of such lines (if known), the doctor, embryologist or clinic which generated the embryo, and the like.

In still another aspect, the invention provides a method for providing embryonic stem cell line information to a database operator or a bank operator or an embryonic stem cell line service provider, the method comprising computer-implemented steps of storing, searching and selecting information records in a database based on particular criteria. Such criteria include with limitation the source of the embryos from which the lines were derived (including the doctor, embryologist or clinic), the line generation protocol, reagents used in the protocol (including supplier and lot number information), and the like.

In still another aspect, the invention provides a computer readable medium having computer readable signals stored thereon, the signals defining instructions that, as a result of being executed by a computer, control the computer to perform a process for providing hESC line information, the process comprising acts of storing, searching and selecting information records in a database, according to one or more criteria.

In yet another aspect, the invention provides a hESC line bank comprising a stored sample of a hESC line generated by any of the above methods. In one embodiment, the bank further comprises a stored sample of human feeder cells and/or human serum derived from a biological relation of the hESC line and used to generate the hESC line. In another embodiment, the sample of a hESC line is cryopreserved, and optionally the sample of human feeder cells and/or human serum is also cryopreserved. In some embodiments, human feeder cells and human serum are both cryopreserved. Preferably the bank comprises more than one sample of hESC lines, and optionally for each sample of hESC lines also a sample of human feeder cells and/or human serum. The sample of the hESC line and the sample of human feeder cells and/or serum may be stored proximally to each other in the bank.

In one embodiment, the bank comprises a plurality of samples of hESC lines. In a related embodiment, a subset or each of the plurality represents a different hESC line. The bank may also comprise a plurality of samples of a given hESC line. In one embodiment, the bank comprises a plurality of samples of human feeder cells and/or human serum. In a related embodiment, a subset or each of the plurality of samples of human feeder cells and/or human serum represents different human feeder cells and/or human serum.

The bank preferably further comprises a database of information records. In one embodiment, the database may comprise an information record for each stored sample of an hESC line. In another embodiment, it may further comprise a second information record for each stored sample of human feeder cells and/or human serum. The information records for the sample of the hESC line and the sample of human feeder cells and/or serum may be associated with each other through an identical information code, but the database is not so limited.

The hESC lines may be generated by a method comprising culturing, on human feeder cells and/or human serum, a human blastocyst generated using a female donor egg, to generate a hESC line, wherein the human feeder cells and/or serum are derived from a biological or genetic relation of the blastocyst and wherein preferably the feeder cells are mitotically inactivated. In some instances, the hESC lines may also be generated using generic feeder cells and preferably such generic feeder cells are human cells having undetectable foreign pathogen content.

In one embodiment, the hESC are generated by culturing inner cell mass (ICM) cells from the human blastocyst on and/or in the presence of the human feeder cells and/or human serum, growing stem cell-like colonies from the ICM cells on and/or in the presence of the human feeder cells and/or human serum, and isolating and culturing cells from the stem cell-like colonies on and/or in the presence of the human feeder cells and/or human serum.

In one embodiment, the human blastocyst is generated from a zygote made by fertilizing an female donor egg with a male donor sperm. In another embodiment, the human blastocyst is generated by somatic cell nuclear transfer into an activated oocyte. In yet another embodiment, the human blastocyst is derived from a split embryo.

In one embodiment, the human blastocyst is generated by culturing a zygote (or an activated oocyte) in the presence of endometrial epithelial cells. In important embodiments, the endometrial epithelial cells are derived from the female egg donor or from another biological or genetic female relation of the blastocyst.

The human feeder cells and human serum may be derived from a genetic mother (e.g., a female egg donor), a genetic father (e.g., male sperm donor (in the case of a zygote)), a biological non-genetic mother, a genetic sibling, or a nucleus donor. The feeder cells may also derive from a non-biological, non-genetic source, in which case, they are referred to as “generic” feeder cells.

Feeder cells derived from female subjects include but are not limited to amniotic epithelial cells, breast fibroblasts, endometrial epithelial cells, endometrial stromal cells, fallopian tube fibroblasts, granulosa cells, oviduct fibroblasts, and placental fibroblasts. Feeder cells derived from male subjects include but are not limited to foreskin fibroblasts. Feeder cells derived from either female or male subjects include but are not limited to lung fibroblasts, oral fibroblasts, skin fibroblasts, bone marrow cells, muscle cells, and other tissue derived stromal cells or fibroblasts.

In one embodiment, the hESC line is generated using serum-free culture conditions. In another embodiment, the hESC line is generated using feeder-free conditions. In still another embodiment, the hESC line is generated using both human feeder cells and human serum. In yet another embodiment, the hESC line is generated under hypoxic (i.e., low oxygen) conditions. Hypoxic conditions are culture conditions having at least 2% but less than 20% oxygen content. Depending on the embodiment, oxygen content may be less than 20%, equal to or less than 15%, equal to or less than 10%, equal to or less than 9%, equal to or less than 8%, equal to or less than 7%, equal to or less than 6%, or equal to or less than 5%, provided that it is equal to or greater than 2%. In some embodiments, oxygen content is greater than 5%.

The method may further comprise cryopreserving the hESC line, and optionally the human feeder cells and/or human serum. The hESC line, the human feeder cells, and/or the human serum may be cryopreserved separately or together.

According to another aspect, the invention provides a hESC line generated by any of the above methods, as well as cultures and progeny thereof.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a block diagram illustrating an example of a computer system on which some embodiments of the invention may be implemented; and

FIG. 2 is a block diagram illustrating an example of a storage system that may be used as part of the computer system to implement some embodiments of the invention.

It is to be understood that the drawings are not required for enablement of the invention.

### DETAILED DESCRIPTION OF THE INVENTION

The invention relates broadly to the generation and maintenance of customized hESC (and lines thereof). As used herein, a customized hESC is a human embryonic stem cell that is intended for use in a particular human recipient (or subset of recipients) and is generated using egg, feeder cells and/or serum, and optionally sperm, from donors who are related to the future intended recipient. The future intended recipient may be pre-existing at the time of hESC line generation, or it may have yet to be born. The recipients are identified or identifiable and all are genetically related to the hESC line. In all embodiments recited herein, humans are preferred as donors and recipients.

The invention relates more specifically to the generation and/or maintenance of hESCs and lines thereof under conditions that facilitate their future use. In particular, the hESC lines are generated using human feeder cells and/or serum derived (i.e., harvested) from someone who is biologically related to the embryo. The use of the human feeder cells and/or serum from a biologically related subject reduces and preferably avoids transmission of new pathogens into the stem cell line from the feeder cells and/or serum. That is, if the feeder cells and/or serum are derived from the genetic mother, father or sibling, the biological non-genetic mother, or the nucleus donor, then there is less likelihood that the embryo or blastocyst will be exposed to new pathogens during the generation and culture period from the feeder cells and/or serum than would be the case if the feeder cells and/or serum were from an unrelated third party or from another species entirely. This is important since it can eliminate the need to screen the stem cell lines when used in the future for the existence of some or all foreign pathogens. This in turn will expedite the future use of the stem cell lines, and increase the interest for such cells.

The invention thus also relates to the future use of hESC lines preferably generated in this manner in genetic relations of the embryo or blastocyst. The existence of a hESC line that is genetically related to various family members, including genetic mothers and fathers and full and half siblings, is advantageous as it may reduce and preferably preclude histo-incompatibility problems in the course of future transplants of cells or tissues into such family members. The invention also contemplates the use of the stem cell lines for future autologous transplant based on the generation of stem cell lines from cloned blastocysts or split embryos.

As used herein, an hESC or line thereof is a human cell or line derived in vitro from an embryo or blastocyst and having stem cell-like properties, as discussed herein. In particular, hESC or lines thereof are considered pluripotent (i.e., able to generate endoderm, mesoderm and ectoderm lineages). The hESC of the invention are not necessarily totipotent (i.e., able to generate another individual). The invention contemplates use of such cells for regeneration of a specific cell lineage(s) or tissue but not of the entire organism. Characteristics of hESC and/or lines thereof are described in greater detail herein. Briefly, these include high nucleus to cytoplasm ratio, prominent nucleoli, the ability to form compact colonies in vitro, expression of markers such as alkaline phosphatase, stage-specific embryonic antigens (SSEA) 3 and 4, TRA 1-60 and TRA 1-81, a normal karyotype (which for humans is 22 pairs of autosomal chromosomes and a pair of sex chromosomes), the ability to develop into mesoderm lineages (e.g., bone, cartilage, smooth muscle, striated muscle and hematopoietic cells), endoderm lineages (e.g., liver, primitive gut and respiratory epithelium) and ectoderm lineages (e.g., neurons, glial cells, hair follicles and tooth buds), immortality as defined by the ability to exist in culture for extended periods of time (e.g., up to a year or more, potentially indefinitely) without differentiating completely and without exhaustion, and/or expression of telomerase activity and the ability to maintain telomere length.

As used herein, the term “zygote” refers to a fertilized egg (i.e., an egg that has been fertilized with a sperm). A zygote is a diploid cell while the unfertilized egg and the sperm are each haploid. The zygote develops into an embryo through several rounds of cell division.

As used herein, the term “embryo” refers to the cell mass that develops from the zygote upon mitosis. The embryos used in the methods of the invention can be freshly prepared or they may be previously cryopreserved. The invention contemplates the use of embryos left over from in vitro fertilization (IVF) procedures for fertility purposes (i.e., surplus embryos) as well as embryos that are generated particularly for stem cell line generation.

As used herein, the term “blastocyst” refers to an organized cell mass of about 150 cells, consisting of a sphere made up of an outer layer of cells called the trophectoderm, a fluid-filled cavity called the blastocoel, and a cluster of cells on the interior called the inner cell mass (ICM). hESC are harvested from disaggregated blastocysts, and more particularly from the inner cell mass of the blastocyst.

The invention is not limited in the source or method of generation of the blastocyst, although it most commonly will derive from processes such as IVF, zygote intra-fallopian transfer (ZIFT), or ovum donation.

IVF generally refers to the process of harvesting female and male sex cells (i.e., eggs and sperm from female and male donors, respectively), fertilizing the egg with the sperm outside the female body, and then implanting the resultant zygote or embryo in the fallopian tubes or uterus of the female body, respectively. The egg may be united with sperm either passively by placing both cells in culture together, or actively by intracytoplasmic sperm injection into the egg.

Ovum donation refers to the harvest of egg cells from a female donor, in vitro fertilization of the egg cells with sperm from a male donor, and implantation of the resultant zygote or embryo into another female. The female into whom the zygote or embryo is implanted is a biological (but not necessarily genetic) relation of the zygote or embryo.

The blastocysts can also be generated using a somatic cell nuclear transfer in which a nucleus from an adult (i.e., non-embryo, non-fetus) somatic (i.e., non-germ cell) cell is harvested and introduced into an enucleated immature cell such as an oocyte. The oocyte is induced into a quasi-fertilized state such that it begins to divide, as would a fertilized egg (i.e., a zygote). Human oocyte activation may be induced for example chemically using for instance the calcium ionophore A23187 (calcimycin) or ionomycin. The resulting activated oocyte develops, as does a zygote, into a blastocyst. As used herein, a blastocyst from a somatic cell nuclear transfer is referred to as a “cloned blastocyst”. The activated oocyte and cloned blastocyst contain a nuclear genetic compliment identical to the donor nucleus and thus any stem cells generated therefrom are potentially autologous to the nucleus donor. (Simerly et al., 2004, Dev Biol 276:237-252; Hwang et al., Science, 303:1669-1674, 2004.)

The generation of stem cell lines from embryos has been described by Thomson in U.S. Pat. Nos. 5,843,780 and 6,200,806. Briefly, zygotes are cultured to the blastocyst stage. It is to be understood that activated oocytes derived from somatic cell nuclear transfer can also be cultured to the blastocyst stage, and stem cell lines generated according to these methods. The ICM may be but need not be isolated. Alternatively, whole blastocysts that have had the zona pellucida removed can also be used. The ICM is harvested, optionally disrupted into cell clusters, and then re-cultured. Generally the ICM and the cells or cell clusters derived therefrom are simply allowed to attach to the solid support in the culture system (e.g., the plate bottom). The ICM-derived cells or cell clusters are then allowed to form colonies or cell masses in vitro. These colonies or cell masses are harvested and disrupted and replated again. Stem cell like colonies formed from these last cultures are then harvested and recultured in order to arrive at stem cell lines. Stem cell like colonies are compact colonies containing cells with high nucleus to cytoplasm ratio and prominent nucleoli.

ESC line generation as described previously, including by Thomson, has employed mouse feeder cells. The concern with this process has been the possibility of pathogen and antigen transfer from the mouse fibroblasts to the human stem cells. Additionally there is always the possibility that some feeder cells are transplanted along with the human stem cells in a transplant setting and this limited (and albeit unwanted) form of xenotransplantation may further expose the transplant recipient to foreign pathogens and antigens, or to unnecessary immune reactions.

The current invention further provides culturing of the embryos or activated oocytes to the point of blastocyst generation in the presence of, for example, human endometrial epithelial cells (Simon et al., 1999, J. Clin Endrocrinol Metab 84:2638-2646; Mercader et al. 2003, Fert Sterlit 80:1162-1168). These endometrial cells may be harvested preferably from the biological mother (whether genetic or not) at any point during the follicular and preferably the luteal phase. Thus, as an example, the embryos or activated oocytes are grown on endometrial epithelial cells until the blastocyst stage and the blastocysts are cultured on other human feeder cells and/or human serum to generate and/or maintain the resultant hESC.

The embryonic stem cell line may be generated and/or maintained on human feeder cells. Feeder cells are generally adherent cells that according to the invention are co-cultured with zygotes, activated oocytes, embryos, blastocysts and/or embryonic stem cells and which support the generation and maintenance of embryonic stem cell lines.

The invention further contemplates the use of feeder cells from non-biological, non-genetic subjects. Such feeder cells are referred to herein as “generic” feeder cells. Preferably, such cells are human in origin. These cells preferably have been screened for the presence of foreign pathogens and have been determined to have undetectable levels of such pathogens. Pathogen screening may be performed using molecular biology techniques such as PCR or immunobiology techniques such as immunohistochemistry or ELISAs.

Although not intending to limit the scope of the invention, it is believed that feeder cells function by providing soluble, extracellular matrix (e.g., insoluble factors deposited by feeder cells) and/or cell bound growth factors essential to stem cell growth and maintenance. They may also provide certain other soluble, extracellular matrix or cell surface molecules that play a role in maintaining the immature state of stem cells.

Feeder cells derived specifically from a female include amniotic epithelial cells (preferably harvested at term) breast fibroblasts (e.g., such as those harvested during reduction mammoplasty), endometrial epithelial cells, endometrial stromal cells, fallopian tube fibroblasts, granulosa cells (preferably harvested after oocyte retrieval), oviduct fibroblasts, and placental fibroblasts. If the feeder cells are endometrial feeder cells, they are preferably harvested from a female subject undergoing IVF prior to stimulation. Feeder cells derived specifically from a male relation include foreskin fibroblasts. Feeder cells derived from a relation of either sex include lung fibroblasts, oral fibroblasts, skin fibroblasts, or other tissue derived stromal cells or fibroblasts, as well as other tissue derived cells such as muscle cells and bone marrow cells.

The feeder cells and/or human serum is preferably from a biological relation, including a biological mother (whether also the genetic mother or not), a genetic sibling, or a nucleus donor. If the hESC lines is generated using an embryo derived from an egg donor and a sperm donor, then the feeder cells may derive from the egg donor, the sperm donor, a biological mother who is not the egg donor (if this involves an ovum donation procedure), or a genetic child of the egg or sperm donor. Preferably, the source of the feeder cells is the egg donor, the sperm donor, the biological non-genetic mother, or a genetic child of both the egg and sperm donor or both the biological, non-genetic mother and the sperm donor.

If the embryo is derived from an IVF procedure involving an egg donor and a sperm donor (e.g., where other embryos so generated are implanted in the egg donor), then the feeder cells and/or serum may be obtained from inter alia, the egg donor. If the embryo is derived from an IVF procedure involving an egg donor and a sperm donor (e.g., where other embryos so generated are implanted in a biological mother who is not the egg donor), then the feeder cells or serum may be obtained from the biological non-genetic mother.

The feeder cells are harvested and grown in culture in order to generate large numbers of these cells. Once a sufficient number have been grown by repeated culturing and splitting, the cells are optionally mitotically inactivated and stored for later use. Mitotic inactivation means that the cells are treated in a manner that prevents them from dividing further but that is not necessarily cytotoxic to the cells. Thus the cells can continue to produce factors necessary for stem cell generation and maintenance even though they are incapable of cell division. Before or after being mitotically inactivated, the feeder cells can be cryopreserved (i.e., frozen) for future use in appropriate aliquots. Mitotic inactivation of feeder cells can be accomplished by ultraviolet (UV), X-, or gamma-irradiation (e.g., at 10-50 Gy), or by chemical means such as senescence inducing drugs (e.g., mitomycin C, toyocamycin, tertbutylhydroperoxide (t-BHP) and hydrogen peroxide (H₂O₂)).

In another aspect, the invention provides methods of generating and maintaining hESC lines in the absence of feeder cells. Such conditions are referred to as “feeder-free”.

Use of human serum for the growth of hESC under feeder-free conditions is described by Stojkovic et al. (Stem Cells Express, online publication May 11, 2005). The method involves coating plates with human serum (derived from clotted blood) for 1 hour at room temperature, followed by removal of excess serum and drying of plates for 1 hour at room temperature. Replating of hESC should also use serum precoated plates. In some embodiments, the human serum is used together with conditioned medium from cultures of fibroblast-like cells derived from spontaneously differentiated human hESC, as described by Stojkovic et al. Thus in some embodiments, hESC lines are generated and/or maintained using any combination of human feeder cells, human serum and conditioned medium from cultures of hESC derived fibroblasts.

Feeder-free conditions further contemplate the use of conditioned medium preferably derived from separate culture of, for example, feeder cells derived from biological relations. Such conditioned medium can be generated by incubation of feeder cells with standard media, and preferably ESC media, available from commercial sources such as Gibco and Invitrogen. Examples include DMEM, IMDM, Knockout serum replacement media (see WO 98/30679), and the like. Cells may be incubated for about 24 hours in order to generate the conditioned media.

The culture systems described herein may also be serum-free in some instances. For example, the cultures may comprise feeder cells yet be serum-free. Serum-free cultures can be performed using serum replacements such as Knock-out™ Serum Replacement (Invitrogen).

The media may further comprise fibroblast growth factor (FGF) such as acidic FGF (aFGF or FGF1) or basic FGF (bFGF or FGF2). Other examples include FGF1beta, FGF3, FGF4, FGF5, FGF6, FGF7, FGF8, FGF9, FGF10, FGF16, FGF17, FGF18, FGF19 and FGF20.

In addition to the foregoing, the hESC lines may be generated and/or maintained under hypoxic conditions. As used herein, “hypoxic conditions” mean an oxygen content of 2% to less than 20%, including any integer therebetween as if explicitly recited herein. Thus the oxygen content may be less than 20%, 19%, 18%, 17%, 16%, 15%, 14%, 13%, 12%, 11%, 10%, 9%, 8%, 7%, 6%, 5%, 4% or 3% provided it is not below 2%. Alternatively, the oxygen content may be 2%-19%, 2%-15%, 2%-10%, 2%-7.5% or 2.5%-7.5%. In some embodiments, oxygen content is 2.5%-10%. In other embodiments, oxygen content is about 5% (i.e., 5%⁺/−0.5%). In still other embodiments, oxygen content is above 5% to less than 20%.

Biological relations are generally familial relations of the embryo, activated oocyte or blastocyst. They include persons genetically related to the embryo, such as for example a parent or a sibling. Biological relations also include females into whom the embryo would have been implanted regardless of whether that female is genetically related to the embryo, provided such a female is a biological mother to a sibling of the embryo, such as for example a female implanted with an embryo derived from ovum donation. This latter class of females are referred to herein as biological, non-genetic mothers. Although they generally are not expected to benefit from the future use of the hESC lines, they can nevertheless contribute feeder cells and/or serum for growing the stem cell lines.

As used herein, the term “genetic relation” refers to individuals who are genetically related to the human stem cell line. That is, the individual may be the donor of the egg or sperm that contributed to making the embryo or the activated oocyte, or the individual may be a genetic child of one or both donors (i.e., a half or full sibling of the embryo, respectively). As discussed in greater detail herein, the use of genetically related hESC increases the chances of a successful transplant using the hESC or their differentiated progeny.

The invention thus also relates generally to the future use of hESC lines, preferably generated according to the invention, in genetic relations of the embryo. The existence of a stem cell line that is genetically related to various family members, including genetic mothers and fathers and full and half siblings, is advantageous as it may reduce and preferably preclude histo-incompatibility problems in the course of future transplants of cells or tissues into certain family members. The invention also contemplates the use of the stem cell lines for future autologous transplant based on the generation of stem cell lines from cloned blastocysts or split embryos.

The sources of egg and sperm (or adult somatic cell nucleus) that make up the embryo will determine the scope of the future use of the stem cell lines derived therefrom and the range of recipients who would benefit as recipients of such cells. IVF or ZIFT processes commonly use egg and sperm from the male and female seeking to be the “familial parents” of the embryo. “Familial parents” as used herein are the parents who would raise the children deriving from the embryo. These processes however can also be carried out using eggs from a third party female and/or sperm from a third party male. The resulting stem cell lines can be used in the future in subjects that are genetically related to the embryo from which they derived. If the familial mother is the biological but not genetic mother, then the stem cell lines may be used in the future for the benefit of the familial genetic father and any children of the familial genetic father. It is unlikely they would be suitable for transplant into the biological, non-genetic mother.

The invention provides for future use of the human hESC lines within months or years after the establishment of the cell line. The stem cell lines therefore may be stored indefinitely such as by cryopreservation. Methods for cryopreserving hESC lines are known in the art and have been described in Ji L, De Pablo J J, Palecek S P: Cryopreservation of adherent human embryonic stem cells Biotechnol Bioeng (2004), 5:299-312; M. Richards, Chui-Yee Fong, Shawna Tan, Woon-Khiong Chan, A. Bongso: An efficient and safe xeno-free cryopreservation method for the storage of human embryonic stem cells. Stem Cells (2004); 22:779-789; Reubinoff B E, Pera M F, Vajta G et al.: Effective cryopreservation of human embryonic stem cells by the open pulled straw vitrification method. Human Reprod. 2001; 10:2182194.

It is to be understood however that in some embodiments the stem cell line may be used prior to cryopreservation, and directly from culture. The invention is not limited in this manner. Preferably, the feeder cells and/or serum are also stored, preferably by cryopreservation. These latter samples can be used when the stem cell line is thawed out for the purpose of genotyping, karyotyping, haplotyping, expansion and/or use (e.g., in vitro or in vivo differentiation).

It is to be understood that one of the benefits provided by the stem cell line generation methods of the invention is the reduction and preferably avoidance of contamination of the stem cells with pathogens or antigens foreign to these cells. This type of contamination can occur when feeder cells from a different species or from a different and unrelated individual of the same species are used. The methods of the invention are intended to avoid as much as possible further testing of the stem cell lines (including testing for pathogen content). In some embodiments, it is anticipated that the human stem cells and/or their differentiated progeny can be transplanted into a genetically related individual without prior testing for some or all pathogen content.

The cell lines may be tested prior to or after cryopreservation for their genotype and histocompatibility haplotype, as appropriate. Genotype testing refers to determining the genotype of the stem cell lines at one or more resolution levels. It is not necessary to determine the genotype of the cell line at single nucleotide resolution. Rather, the genotyping must only be carried out at a resolution level that allows one of ordinary skill to determine the similarity between the stem cell line and any intended recipient thereof. Genotyping can be carried out in a number of ways including but not limited to restriction fragment length polymorphism (RFLP).

The cell lines and/or their progeny can also be tested for their histocompatibility haplotype. A histocompatibility haplotype is a set of alleles at the histocompatibility gene loci that is used by the immune system to distinguish between self and non-self (i.e., foreign) tissues and/or cells. In humans, the major histocompatibility (MHC) locus is composed of four loci on the short arm of chromosome 6. Humans also have a set of minor histocompatibility loci. As an example, human leukocyte antigen (HLA) typing is commonly performed for various transplants such as hematopoietic cell transplants. Major and minor histocompatibility antigens are present on cell surfaces and are recognized by the immune system as an indicator of the origin of the cell or tissue. Cells or tissues that are viewed as foreign will usually be rejected by the recipient via a host versus graft immune response. However, it is sometimes possible to overcome some differences in histocompatibility, particularly those in the minor histocompatibility loci, using for example immunosuppressive drugs such as but not limited to cyclosporin A, FK506, rapamycin, cyclophosphamide (CY), procarbazine (PCB), and antithymocyte globulin (ATG). Additionally, certain tissues are less susceptible to differences at for example the HLA loci in humans. These tissues include but are not limited to liver, kidney, and the central nervous system. It has recently been reported that embryonic stem cells possess immune privileged properties (Li et al. Stem Cells 2004 33:448-456).

Certain embodiments of the invention also contemplate the use of hESC for autologous transplant into a recipient. The recipient in these instances may be a human subject from whom a somatic cell has been harvested and used to generate the cloned blastocyst that gave rise to the hESC line.

Alternatively, the recipient in these instances may be a human being derived from a split embryo. Splitting of embryos has been carried out successfully in the mouse system. The Examples describe a process for splitting of embryos in greater detail. Briefly, the embryo is split into two portions, one of which develops into the fetus and ultimately the human recipient, and the other of which is used for the generation of the stem cell line.

In either situation, the recipient of the hESC line and progeny thereof are genetically identical to the stem cell line, and the stem cell line can be used to treat the individual of virtually any condition benefiting from a cell transplant, as described in greater detail herein. Such transplants are referred to herein as autologous transplants as they are between cells and recipients that are identical by all measures including genotype and haplotype.

The hESC lines can be used in both research and therapeutic applications. The lines can be differentiated into a number of lineages including but not limited to endothelial cells, neurons, hematopoietic cells, cardiomyocytes, skeletal muscles, hepatocytes, insulin-producing cells, glial progenitor cells, osteoblasts, gametes and kidney cells. Differentiation can occur in vitro or in vivo, depending on the application. In vitro differentiation of hESC will allow the study of organ and/or tissue formation. Should the cell lines be found to harbor a particular genetic mutation (or more), then these lines may be differentiated in vitro to determine the effect of such mutation on organ and/or tissue development.

Accordingly, the stem cell lines can be used in a transplant setting in the treatment (including prevention) of various conditions including but not limited to Parkinson's disease (dopaminergic neurons), Alzheimer's disease (neural precursors), Huntington's disease (GABAergic neurons), blood disorders such as leukemia, lymphoma myeloma and anemia (hematopoietic cells), side-effects of radiation e.g., in transplant patients (hematopoietic precursors), myocardial infarction, ischemic cardiac tissue or heart-failure (partially- or fully-differentiated cardiomyocytes), muscular dystrophy (skeletal muscle cells), liver cirrhosis or failure (hepatocytes), chronic hepatitis (hepatocytes), diabetes including type I diabetes (insulin-producing cells such as islet cells), ischemic brain damage (neurons), spinal cord injury (glial progenitor cells and motor neurons), amyotrophic lateral sclerosis (ALS) (motor neurons), orthopedic tissue injury (osteoblasts), kidney disease (kidney cells), corneal scarring (corneal stem cells), cartilage damage (chondrocytes), bone damage (osteogenic cells including osteocytes), osteoarthritis (chondrocytes), myelination disorders such as Pelizaeus-Merzbacher disease, multiple sclerosis, adenoleukodystrophies, neuritis and neuropathies (oligodendrocytes), and hair loss. References documenting the differentiation of ESC into these various lineages include Bjorklund et al., 2002, PNAS USA 99:2344-2349 (dopaminergic neurons), West and Daley, 2004, Curr Opin Cell Biol 16:688-692; U.S. Pat. No. 6,534,052 B1; Kehat and Gepstein, 2003, 8:229-236; Nir et al., 2003, 58:313-323; U.S. Pat. Nos. 6,613,568 and 6,833,269. In vitro as well as in vivo differentiation is contemplated by the invention. Thus, transplant of differentiated cells and/or undifferentiated or partly differentiated hESC is embraced by the invention.

The invention also contemplates the ability to transduce hESC or their differentiated progeny with particular nucleic acids. This may be done prior to transplant into a genetically related individual. For example, if the hESC are known to harbor a genetic mutation identical to that in the recipient of these cells, then simply transplanting the hESC or their progeny as is may not be therapeutically useful. However, if the genetic mutation is corrected by the transduced nucleic acid, then the hESC and/or their progeny are more likely to be suitable therapeutic agents.

The invention provides yet another use for the human hESC generated according to the methods described herein. Based on the methods provided herein, it will be possible to create stem cell lines that are genetically related to a human being. These cells can therefore be used to screen various agents for toxicity and in some embodiments therapeutic efficacy. The readouts from such in vitro assays are correlative of the in vivo toxicity or efficacy such agents would exhibit in the human subjects. Thus, the effect of the agent on the differentiated progeny of hESC in vitro is a form of surrogate marker or readout for how the agent will function in vivo in the human subject. Using this technique it should be possible to customize a therapy for an individual by distinguishing agents that are safe and efficacious from those that are not.

It is expected that the human subject that is the intended recipient of the stem cells or their progeny will have or be at risk of developing a condition that affects one or more known cell lineages. The hESC are therefore preferably differentiated into those cell lineages and those differentiated progeny are then exposed to the agent. As described herein, it is now possible to differentiate hESC into various cell lineages including but not limited to melanocytes, hematopoietic cells, hepatocytes, kidney cells, skeletal muscle cells, dopaminergic neurons, glial cells, cardiomyocytes, endothelial cells, and osteoblasts. Thus for example a subject that has or is at risk of developing leukemia would want to screen differentiated hematopoietic cells for their response profile to one or more anti-leukemia agents. As another example, a subject having muscular dystrophy would want to screen differentiated skeletal muscle cells for their response to one or more agents intended for use in muscular dystrophy.

In one embodiment, the response profile data so generated can be stored on computer readable media, correlated with corresponding parameter values (such as particular agent, dose thereof, and degree and quality of response), processed to determine optimal values and can then be reported to a client (or medical practitioner) in computer readable or human readable format.

The agents to be tested include those used clinically as well as experimental agents.

In some more common embodiments, such testing will focus on the cytotoxicity of drugs in particular differentiated progeny of the hESC. Accordingly, in these assays, the readout would be cell death (or conversely cell viability).

Drugs that can be tested according to these methods particularly for whether they are toxic to cells of a particular genetic background include but are not limited to adrenergic agent; adrenocortical steroid; adrenocortical suppressant; aldosterone antagonist; anabolic; analeptic; analgesic; androgen; anesthesia, adjunct to; anesthetic; anorectic; anterior pituitary suppressant; anti-acne agent; anti-adrenergic; anti-allergic; anti-androgen; anti-anemic; anti-anginal; anti-arthritic; anti-asthmatic; anti-atherosclerotic; anticholelithic; anticholelithogenic; anticholinergic; anticoagulant; anticoccidal; anticonvulsant; antidepressant; antidiabetic; antidiarrheal; antidiuretic; anti-emetic; anti-epileptic; anti-estrogen; antifibrinolytic; antiglaucoma agent; antihemophilic; antihemorrhagic; antihistamine; antihyperlipidemia; antihyperlipoproteinemic; antihypertensive; antihypotensive; anti-inflammatory; antikeratinizing agent; antimigraine; antimitotic; antimycotic, antinauseant, antineoplastic, antineutropenic, antiparkinsonian; antiperistaltic, antipneumocystic; antiproliferative; antiprostatic hypertrophy; antipruritic; antipsychotic; antirheumatic; antiseborrheic; antisecretory; antispasmodic; antithrombotic; antitussive; anti-ulcerative; anti-urolithic; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; cognition adjuvant; cognition enhancer; depressant; diagnostic aid; diuretic; dopaminergic agent; ectoparasiticide; emetic; enzyme inhibitor; estrogen; fibrinolytic; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; immunomodulator; immunoregulator; immunostimulant; immunosuppressant; impotence therapy adjunct; keratolytic; LHRH agonist; liver disorder treatment; luteolysin; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; progestin; prostaglandin; prostate growth inhibitor; prothyrotropin; psychotropic; pulmonary surface; relaxant; repartitioning agent; scabicide; sclerosing agent; sedative; sedative-hypnotic; selective adenosine A1 antagonist; serotonin antagonist; serotonin inhibitor; serotonin receptor antagonist; steroid; symptomatic multiple sclerosis; thyroid hormone; thyroid inhibitor; thyromimetic; tranquilizer; amyotrophic lateral sclerosis agent; cerebral ischemia agent; Paget's disease agent; unstable angina agent; uricosuric; vasoconstrictor; vasodilator; wound healing agent; xanthine oxidase inhibitor. Those of ordinary skill in the art will know or be able to identify agents that fall within any of these categories, particularly with reference to the Physician's Desk Reference.

In still other aspects, the invention provides banks of hESC lines. A hESC line bank is a physical collection of one or more hESC line samples. Such banks preferably contain more than one sample (i.e., aliquot) of a hESC line and more preferably such samples correspond to different hESC lines. However, the bank may contain more than one aliquot of a given hESC line. The hESC lines may be generated according to the methods of the invention, in which case the bank may also contain one or more samples of the human feeder cells and/or human serum used to generate the hESC lines. The hESC lines and/or human feeder cells and/or human serum are preferably stored in a cryopreserved form as described herein.

The banks are primarily intended as family banks in that their samples are intended primarily for the use of clients that contract for the generation and/or storage of hESC line samples, or their family members. However, with permission from the client, a particular sample in the bank may be made available to the public at large, in which case it may be transferred to a public bank or its information record will be made available either in whole or in part to third parties involved in transplantation services, such as the Red Cross.

The hESC line samples, human feeder cells and/or human serum samples may be stored proximally or distally to each other, depending, for example, on the exact cryopreservation environment required for maximum viability and/or integrity of each sample type.

It is expected that storage of stem cell line samples in a bank will be performed for a fee that, for example, may be dependent on the length of storage time and/or number of stored samples.

The bank also preferably comprises a database, preferably stored in one or more computer-readable media, that contains information for each stored sample. The database may have any suitable structure. For example, it may have one or more flat files or it may include a relational structure based on tables. A basic database may be organized into one or more information records for each stored sample. Each information record may be organized into one or more fields, each having one or more subfields. The fields may be coded according to the sensitivity of information contained therein and thus the degree of accessibility of the information stored therein. Subfields may be similarly categorized. Access controls may be applied to records or fields so that only authorized personnel may access them. With proper access controls (i.e., user authorization mechanism), the authorization attributes of a user may control the degree to which the user may search the database. A person with full clearance, such as the database operator, may be able to access all fields and subfields. Other persons, however, may have clearance only for one or a subset of fields and/or subfields. For example, the hESC line service provider (i.e., the person that is generating the hESC line sample) may have access to fields relating to characterization of the lines, including protocol specifics and generation outcomes. In this way, the hESC line service provider can retrospectively assess the efficacy of the protocols and reagents used in hESC line derivation.

If a relational database structure is used, the information for a particular record may be dispersed among a number of different tables—e.g., one table per field or subfield—for all or portions of the database, with entries linked by an identification code or index for the record.

The fields may be categorized in any number of ways. In one non-limiting example, there are at least five fields per information record. A first field contains information, preferably organized in subfields, relating to identifiers of the sample. This field contains information that is the most easily accessible and least privileged of all the information stored for a given sample. Such information should be sufficient to determine presence of a stored sample within the bank collection. Subfields may therefore correspond to identification codes (or deposit numbers), number of sample aliquots stored, the date of generation of the hESC line, the date of storage of the sample, and the like.

In this same example, a second field contains information, preferably organized in subfields, relating to sample particulars. This field contains information that is more privileged, and therefore less accessible, than the information of the first field. Such information should be sufficient to locate a stored sample with a bank collection. Subfields may correspond, for example, to the physical location of the sample in the bank, the existence of a feeder cell and/or serum sample corresponding to the hESC line sample, identification code (or deposit number) of the feeder cell and/or serum sample, physical location of the feeder cell and/or serum sample, and the like.

A third field contains additional sample information relating to the phenotype, genotype, pathogen profile and growth characteristics of the sample. This field contains information that may be even more privileged and less accessible than that of the second field. Such information, for example, should be sufficient to allow someone to confirm the identity of the sample prior to its use. This information can also be used to determine if one or more sample characteristics have changed as a result of storage. Subfields may correspond, for example, to the haplotype, genotype, and karyotype of the sample, passage number of the stored sample, growth characteristics such as proliferation rate or doubling time, phenotype of the sample for example according to a set of stem cell markers including but not limited to TRAs, SSEAs, alkaline phosphatase, and the like, growth conditions including the supplier and lot numbers for reagents, and the like.

A fourth field contains information, preferably organized in subfields, relating to sample generation protocols. This field may be accessible to the hESC line service provider. Such information can be used to analyze efficacy of derivation and culture protocols in a retrospective manner. Subfields may, for example, correspond to the doctor, embryologist and/or clinic that derived the embryo, the length and manner of storage of the embryo prior to hESC line derivation, the freezing date of the embryos, the freezing protocol, the manner of transfer of the embryo, the stage of the embryos when thawed, the quality of the thawed embryos, the number of blastocysts derived from each thawed embryo, the derivation protocol, the reagents used in the derivation protocol including the supplier, catalog number and lot number, and the like.

A fifth field contains information, preferably organized in subfields, relating to the client, owner and source of the samples. This field contains the most privileged information of all the information stored for a given sample. This information may be used to identify and locate samples should the client lose the identification code. It may also be used to store personal information including name and contact information of the egg donor, the sperm donor and/or the biological non-genetic mother, familial and/or genetic relations of both including full and half “siblings” of the stored line, the karyotype, phenotype and genotype of the egg and sperm donor, the pathogen profiles of the egg donor, sperm donor and/or biological non-genetic mother, the pathogen profiles of the feeder cell donor and/or the serum donor, and the like.

Examples of fields therefore may be a high level sample identification field, a feeder or serum field, a sample phenotype field, a sample genotype field, a sample derivation field, a donor field, a client field, and the like.

It is to be understood that the preceding is only one exemplification of the invention and that databases may be set up using any number of fields and subfields and/or tables, and any number of clearance levels. Those of ordinary skill in the art will be able to set up comparable databases using the teachings provided herein. For example, some databases may comprise a plurality of fields rather than adopting a field and subfield hierarchy. Other databases may comprise only a subset of the fields and subfields listed above, and may optionally comprise additional fields of use to the database operator or other user of the database.

Certain fields and subfields may be static fields. A static field is one which, once correctly populated, cannot be changed. Examples of static fields in the database include identification code (or deposit number), date of hESC derivation, names of egg and sperm donors, doctors, embryologists and clinics which derived the embryos, date of storage of the samples, and the like. Other fields and subfields are non-static. A non-static field is a field which once populated can be changed. Examples of non-static field include relations of the egg or sperm donor, pathogen content of the egg or sperm donors, status of samples, disposition of samples, and the like. The database preferably will also maintain a revision history for each field and subfield so that the operator can review updates and the dates of such updates, the identity of the operator who changed the information, etc.

Fields and subfields that may be integrated into the database therefore include, but are not limited to, deposit number (static field), location of deposit in the bank, name of sperm donor (static field), name of egg donor (static field), relations of sperm donor (non-static field), a family tree for either donor or a combined tree of both, relations of egg donor (non-static field), medical history of egg donor, sperm donor and/or biological non-genetic mother (non-static field), family medical history of egg donor, sperm donor and/or biological non-genetic mother (non-static field), full genetic siblings of the line (non-static field), half siblings of the line (non-static field), source of the embryo including doctor, embryologist and/or IVF clinic (static field), date of cell line derivation (static field), derivation conditions (static field), passage number (static field), growth conditions (static field), haplotype/genotype/karyotype of the line (static field), growth characteristics of the line (static field), phenotype of the line (static field), pathogen testing of line and/or donors, contact information for “owners” of line, feeder cells or human serum used in derivation/propagation (static field), feeder cell or serum identity (static field), location, source and growth conditions, number of aliquots stored for each deposit, status of deposit, disposition of deposit, results of disposition (e.g., differentiation characteristics, use, etc.), differentiation capacity of the cells line (e.g., measured as the efficiency to form embryoid bodies), percent of pluripotent, undifferentiated cells in culture as measured by Oct 3/4, Nanog, Tra 1-60, Tra 1-81 and/or SSEA4 expression, proliferation rate/doubling time, date when line was cryopreserved, lot numbers and suppliers of media and other reagents used for derivation, number of embryos used for ES cell generation, number of blastocysts developed from the thawed embryos, embryo quality of thawed embryos, stage of the thawed embryos (Day 1—pronuclear, Day 3, blastocyst), freezing date of the embryos provided by the IVF clinic, image files that document all stages of ES cell derivation and embryo, quality after thawing, efficiency of hESC line derivation, scanned copy of signed patient consent form, and any other miscellaneous fields or subfields deemed appropriate by the bank or database operator or the hESC line service provider.

The invention embraces manipulation of the database information by one or optionally more than one user, provided such user has the requisite clearance. The database can be further structured such that access to a given field (or subfield) will allow access only to other fields (or subfields) at the same level of clearance. In another instance, certain users may be given access to a select number of fields (or subfields) for a particular purpose and potentially for a limited time, regardless of the clearance level of such fields and subfields. For example, the hESC line service provider may perform a retrospective analysis of hESC line derivation using a select number of fields or subfields. Accordingly, the database can be structured so that access to a particular subfield does not make an entire field accessible also.

The database may be searched according to any number of fields or subfields, either singly or in combination, provided the operator has the requisite clearance. An example of a query is a particular identification code (or deposit number) and particulars regarding any associated feeder cell or serum sample present in the database.

An example of a search is one that calls for records having a hESC line generation efficiency that is greater than 25% followed by one that calls for any single or combination of parameters relating to the embryo, the embryo source, the derivation protocol, and the like. In this manner, an operator can determine which parameters (and information for such parameters) correlate with better derivation efficiency. Another example of a search is one that calls for records showing a particular identification number, followed by a search for feeder cells or serum samples associated with that line, optionally followed by the location of both, and further optionally followed by the growth conditions for the line. This may represent a common search of the bank, particularly as a client comes back to the bank for use of the deposited line. Still another example of a search is one in which a future relation of the line contacts the bank requesting access and use of the deposited line. This may occur after the death of the client, donors, etc. provided that the client, donors, etc. have agreed to such future use. The search may call for records having the name of the requestor and or relation of the requestor in one or more fields of the record. For example, the requester may be a child or a grandchild of the egg and sperm donor. If a child, it is likely that the record will contain the name of such individual and the search may be conducted using that information. If a grandchild, it may be less likely that the record will contain the name of such individual and the search would be conducted for example on the name of that individual's mother or father. Yet another search may call for records having a particular donor's name followed by a search for the consent form. Still another search may call for records having a particular identification record, followed by a download of all information relating to the stored sample to be forwarded to a third party including the growth characteristics and conditions of the deposit, the image file of colonies of the line, the karyotype (and optionally image thereof) of the line, the phenotype of the line, the pathogen testing results performed on the line, and the like. It is possible that not all information within the information record for a sample is ultimately transferred to a third party once request for transfer of the line is effected. This is because some of the fields and/or subfields within an information record may be for internal use or the bank or database operator or the hESC line service provider.

It is to be understood that the database can be searched based on the physical holdings (or content) of a bank as well as based on the information stored in the database itself without the need for retrieval of any sample and optionally independent of sample identifier information or even current sample presence in the bank. Accordingly, it is possible that the bank may maintain information records in whole or in part relating to samples that no longer exist in the bank.

It is further to be understood that storing and searching identification records and tracking samples within the bank is not entirely dependent on a computer-based method, although it is in some instances the method of choice due to its flexibility and convenience. When a computer-based method is used, some or all of the data may be encrypted using any acceptable cryptographic system, to protect the privacy and security of the data.

Thus, the use of the stem cell line bank may be computer implemented. Software and data can be stored on computer readable media and can be executed or accessed at a later date. Software can be used, for example, to obtain data, store data, organize data, correlate data and to provide information to a client, the bank operator, or the hESC generation service provider.

Some of the methods described herein and various embodiments and variations of the methods and acts, individually or in combination, may be defined by computer-readable signals tangibly embodied on more computer-readable media, for example, non-volatile recording media, integrated circuit memory elements, or a combination thereof. Computer readable media can be any available media that can be accessed by a computer. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, other types of volatile and non-volatile memory, any other medium which can be used to store the desired information and which can be accessed by a computer, and any suitable combination of the foregoing. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, wireless media such as acoustic, RF, infrared and other wireless media, other types of communication media, and any suitable combination of the foregoing.

Computer-readable signals embodied on one or more computer-readable media may define instructions, for example, as part of one or more programs, that, as a result of being executed by a computer, instruct the computer to perform one or more of the functions described herein, and/or various embodiments, variations and combinations thereof. Such instructions may be written in any of a plurality of programming languages, for example, Java, Visual Basic, C, C#, or C++, Fortran, Pascal, Eiffel, Basic, COBOL, etc., or any of a variety of combinations thereof. The computer-readable media on which such instructions are embodied may reside on one or more of the components of any of systems described herein or known to those skilled in the art, may be distributed across one or more of such components, and may be in transition therebetween.

The computer-readable media may be transportable such that the instructions stored thereon can be loaded onto any computer system resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement the above-discussed aspects of the present invention.

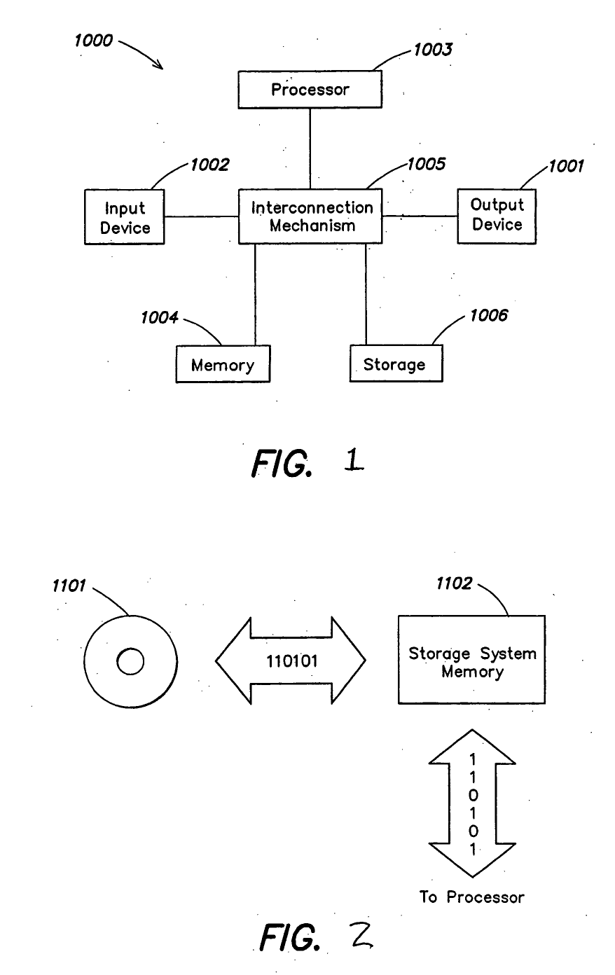

It should be appreciated that any single component or collection of multiple components of a computer system, for example, the computer system described in relation to FIGS. 1 and 2, that perform the functions described herein can be generically considered as one or more controllers that control such functions. The one or more controllers can be implemented in numerous ways, such as with dedicated hardware and/or firmware, using a processor that is programmed using microcode or software to perform the functions recited above or any suitable combination of the foregoing.

Each of the systems described herein and illustrated in FIGS. 1 and/or 2, and components thereof, may be implemented using any of a variety of technologies, including software (e.g., C, C#, C++, Java, or a combination thereof), hardware (e.g., one or more application-specific integrated circuits), firmware (e.g., electrically-programmed memory) or any combination thereof. One or more of the components may reside on a single device (e.g., a computer), or one or more components may reside on separate, discrete devices. Further, each component may be distributed across multiple devices, and one or more of the devices may be interconnected.

Further, on each of the one or more devices that include one or more components of the systems, each of the components may reside in one or more locations on the system. For example, different portions of the components of these systems may reside in different areas of memory (e.g., RAM, ROM, disk, etc.) on the device. Each of such one or more devices may include, among other components, a plurality of known components such as one or more processors, a memory system, a disk storage system, one or more network interfaces, and one or more busses or other internal communication links interconnecting the various components. The systems, and components thereof, may be implemented using a computer system such as that described below in relation to FIGS. 1 and 2.

Various embodiments according to the invention may be implemented on one or more computer systems. These computer systems, may be, for example, general-purpose computers such as those based on Intel PENTIUM-type and XScale-type processors, Motorola PowerPC, Motorola DragonBall, IBM HPC, Sun UltraSPARC, Hewlett-Packard PA-RISC processors, any of a variety of processors available from Advanced Micro Devices (AMD) or any other type of processor. It should be appreciated that one or more of any type of computer system may be used to implement various embodiments of the invention.

A general-purpose computer system according to one embodiment of the invention is configured to perform any of the functions described above. It should be appreciated that the system may perform other functions and the invention is not limited to having any particular function or set of functions.

For example, various aspects of the invention may be implemented as specialized software executing in a general-purpose computer system 1000 such as that shown in FIG. 1. The computer system 1000 may include a processor 1003 connected to one or more memory devices 1004, such as a disk drive, memory, or other device for storing data. Memory 1004 is typically used for storing programs and data during operation of the computer system 1000. Components of computer system 1000 may be coupled by an interconnection mechanism 1005, which may include one or more busses (e.g., between components that are integrated within a same machine) and/or a network (e.g., between components that reside on separate discrete machines). The interconnection mechanism 1005 enables communications (e.g., data, instructions) to be exchanged between system components of system 1000. Computer system 1000 also includes one or more input devices 1002, for example, a keyboard, mouse, trackball, microphone, touch screen, and one or more output devices 1001, for example, a printing device, display screen, speaker. In addition, computer system 1000 may contain one or more interfaces (not shown) that connect computer system 1000 to a communication network (in addition or as an alternative to the interconnection mechanism 1005.

The storage system 1006, shown in greater detail in FIG. 2, typically includes a computer readable and writeable nonvolatile recording medium 1101 in which signals are stored that define a program to be executed by the processor or information stored on or in the medium 1101 to be processed by the program. The medium may, for example, be a disk or flash memory. Typically, in operation, the processor causes data to be read from the nonvolatile recording medium 1101 into another memory 1102 that allows for faster access to the information by the processor than does the medium 1101. This memory 1102 is typically a volatile, random access memory such as a dynamic random access memory (DRAM) or static memory (SRAM). It may be located in storage system 1006, as shown, or in memory system 1004, not shown. The processor 1003 generally manipulates the data within the integrated circuit memory 1004, 1102 and then copies the data to the medium 1101 after processing is completed. A variety of mechanisms are known for managing data movement between the medium 1101 and the integrated circuit memory element 1004, 1102, and the invention is not limited thereto. The invention is not limited to a particular memory system 1004 or storage system 1006.

The computer system may include specially-programmed, special-purpose hardware, for example, an application-specific integrated circuit (ASIC). Aspects of the invention may be implemented in software, hardware or firmware, or any combination thereof. Further, such methods, acts, systems, system elements and components thereof may be implemented as part of the computer system described above or as an independent component.

Although computer system 1000 is shown by way of example as one type of computer system upon which various aspects of the invention may be practiced, it should be appreciated that aspects of the invention are not limited to being implemented on the computer system as shown in FIG. 1. Various aspects of the invention may be practiced on one or more computers having a different architecture or components than that shown in FIG. 1.

Computer system 1000 may be a general-purpose computer system that is programmable using a high-level computer programming language. Computer system 1000 may be also implemented using specially programmed, special purpose hardware. In computer system 1000, processor 1003 is typically a commercially available processor such as the well-known Pentium class processor available from the Intel Corporation. Many other processors are available. Such a processor usually executes an operating system which may be, for example, the Windows® 95, Windows® 98, Windows NT®, Windows® 2000 (Windows® ME), Windows® XP, Windows CE® or Pocket PC® operating systems available from the Microsoft Corporation, MAC OS® System X available from Apple Computer, the Solaris® Operating System available from Sun Microsystems, Linux available from various sources, UNIX available from various sources or Palm OS available from Palmsource. Many other operating systems may be used.

The processor and operating system together define a computer platform for which application programs in high-level programming languages are written. It should be understood that the invention is not limited to a particular computer system platform, processor, operating system, or network. Also, it should be apparent to those skilled in the art that the present invention is not limited to a specific programming language or computer system. Further, it should be appreciated that other appropriate programming languages and other appropriate computer systems could also be used.

One or more portions of the computer system may be distributed across one or more computer systems (not shown) coupled to a communications network. These computer systems also may be general-purpose computer systems. For example, various aspects of the invention may be distributed among one or more computer systems configured to provide a service (e.g., servers) to one or more client computers, or to perform an overall task as part of a distributed system. For example, various aspects of the invention may be performed on a client-server system that includes components distributed among one or more server systems that perform various functions according to various embodiments of the invention. These components may be executable, intermediate (e.g., IL) or interpreted (e.g., Java) code which communicate over a communication network (e.g., the Internet) using a communication protocol (e.g., TCP/IP).

It should be appreciated that the invention is not limited to executing on any particular system or group of systems. Also, it should be appreciated that the invention is not limited to any particular distributed architecture, network, or communication protocol.

Various embodiments of the present invention may be programmed using an object-oriented programming language, such as SmallTalk, Java, C++, Ada, or C# (C-Sharp). Other object-oriented programming languages may also be used. Alternatively, functional, scripting, and/or logical programming languages may be used. Various aspects of the invention may be implemented in a non-programmed environment (e.g., documents created in HTML, XML or other format that, when viewed in a window of a browser program, render aspects of a graphical-user interface (GUI) or perform other functions). Various aspects of the invention may be implemented as programmed or non-programmed elements, or any combination thereof. Further, various embodiments of the invention may be implemented using Microsoft.NET technology available from Microsoft Corporation.

The invention therefore further contemplates a method for conducting a hESC line generation and storage service. Such a method would comprise a service for generating or accepting embryos from, for example, in vitro fertilization procedures for or from a client, a derivation service for generating hESC lines from the embryos as described herein and/or as described in the art, and an hESC lines (and potentially human feeder cells and/or human serum) storage service such as a cryopreservation service. The service may further comprise a cell differentiation service as well, whereby the hESC lines are differentiated fully or partially towards a particular cell lineage(s). The service may further comprise retrieval of hESC lines stored in, for example, a bank. Retrieval of such lines may be computer-facilitated via database searches and automated sample retrieval. Accordingly, the invention envisions a system in which a database and its search and identification capabilities are integrated with an automated cell retrieving system. The service may further comprise a cell thawing service if the hESC lines are stored in a cryopreserved form. The service may further comprise transfer of the hESC line sample to the client, the owner or a third party. The service may optionally comprise destruction of the hESC line sample should the client so instruct. The method may further include a billing system for billing a client based on the service provided, and such billings may be generated on a weekly, monthly or yearly basis. Billings may be made directly to the client or to an insurance provider.

The invention also provides another method for conducting a hESC line screening service. This method contemplates testing one or more agents for their effect on stored hESC or their differentiated progeny and thereby generating an agent response profile. The method then may supply such profile information to a medical practitioner or to the client. Alternatively, the method may involve processing of the profile information in order to formulate a therapeutic regimen suitable for the client and/or the intended recipient of treatment.

The present invention is further illustrated by the following Examples, which in no way should be construed as further limiting.

### EXAMPLES

### Example 1

### Isolation and Generation of Isogenic Feeder Cells

Endometrial biopsies were obtained with consent from the patient with a catheter (Gynetics, Amsterdam, The Netherlands) during the luteal phase of the previous cycle of the IVF process. Endometrial samples were placed in a drop of Dulbecco's Modified Eagle Medium (DMEM) and cleaned with a scalpel, eliminating any clots and mucus. Then using two scalpels in a scissor-like action, the tissue was cut into small pieces measuring less than 1 mm. Once cut, the tissue was transferred to a conical tube containing 10 ml of a 0.1% collagenase IA solution (obtained by mixing 9 ml DMEM with 1 ml collagenase). To carry out the digestion, the Falcon tube was placed horizontally in a shaking water bath at 37° C. for 1 hour. At the end of the incubation, the conical tube was removed, placed in a rack and left to rest for 10 minutes inside a laminar flow cabinet. The supernatant, in which the stromal cells are suspended, was collected and filtered under vacuum through a 30 μm pore-diameter membrane that had previously been sterilized by exposure to UV light for 24 hours. Stromal cells were pelleted by centrifugation at 2000 rpm for 10 minutes and washed with DMEM. The pellet contains stromal and blood cells, and was resuspended with an automatic pipette in 300 μl of a solution containing 4 mg/ml DNase. The reaction was stopped by adding 1 ml of 1% fetal bovine serum (FBS) in 9 ml DMEM. Cells were counted to ensure a suitable seeding density of stromal cells (3×10⁶ per 5 ml). The medium was changed daily during the early stages of culture (e.g., up to passage 2) and every two days thereafter.

Cells were passaged when they reached 70-80% confluence. This was done by washing them twice for 5 minutes each with Hanks' Balanced Salt Solution (HBSS), adding 3 ml trypsin-EDTA (1×), and incubating the mixture for 5 minutes at 37° C. Cells were checked under a microscope to ensure that they had lifted off the plastic and entered suspension. If not, cells were dislodged by tapping the sides of the flask briskly. Trypsin was inactivated by adding 5 ml culture medium containing serum. The cell suspension was collected with a serological pipette, transferred to a 14 ml Falcon tube, and sealed with parafilm. The cells were then centrifuged at 2000 rpm for 10 minutes and the supernatant was discarded, followed by resuspension in 2 ml culture medium. A 100 μl sample was removed for a cell count.

Human endometrial stromal cells after passage 3 were inactivated by H₂O₂, mitomycin C or gamma irradiation to prepare a feeder layer that supports customized hESC derivation. For H₂O₂ inactivation, stromal cells are left to become confluent in the tissue culture dish and then treated for 30-45 minutes with freshly prepared 100 μM H₂O₂ in the fibroblast medium. Upon treatment, medium is replaced with the fresh fibroblast medium. The next day cells are trypsinized and either subcultured or frozen. Five days after thawing or subculturing, cells are growth arrested and ready to serve as feeders.

For irradiation, cells in suspension were collected in 10 ml tubes and sealed with parafilm. The tubes were transported to an electron accelerator in a portable incubator at 37° C. and 5% CO₂. Once in the accelerator, tubes containing the cells to be irradiated were placed on pieces of plasticine inside the tray and the tray was filled with 1.5 litres of sterile water at 37° C. The accelerator was programmed and cells were irradiated at 12 Gy. The cells were replaced in the portable incubator and returned to the laboratory. The cells were then seeded in a flask in culture medium containing 10% FBS and, after 24-48 hours, they were ready for use. The cells could also be stored for future use by freezing.

Irradiated human endometrial stromal cells were seeded into 4- or 6-well plates, as described above, at a density of 50,000 or 100,000 cells per well, respectively, using stromal-cell culture medium (see below). Cells were incubated at 37° C. in an incubator with 5% CO₂ for at least two days, with media being changed every 1-2 days. When the feeders were ready to receive ICM cells, ES cells, or blastocyst, the medium was changed to hES medium (see below) supplemented with 4 ng/ml bFGF. Inner cell mass cells or zona free blastocysts were then added to each plate with hES medium.

### Example 2

### Derivation of Human Embryonic Stem Cells on Isogenic Feeder Cells

a. Production of a Blastocyst:

During development, a zygote (i.e., a fertilized egg) divides to reach the 8-cell stage, which divides twice to yield a 32-cell stage known as a morula. After 5-6 days, the morula undergoes cavitation to form a blastocyst comprising an inner cell mass (ICM) and a layer of trophoectoderm cells which surrounds a fluid-filled cavity known as the blastocoel. The ICM is located within the blastocoel.

Normal and split embryos can develop into blastocysts in vitro. Techniques for achieving this developmental progression are well known in the art including e.g., culture on monolayers. In one method, zygotes are co-cultured up to the blastocyst stage with endometrial epithelial cells (Simon et al. 1999, J. Clin Endocrinol Metabol, 84:2638-2646), which give high efficiency blastocyst development. Zona pellucida is removed from blastocysts by brief incubation in Tyrode's solution, or by the use of pronase or laser dissection.

b. Seeding of Blastocyst on Isogenic Endometrial Feeder and Expansion of Embryonic Stem Cells

Zona free blastocysts are placed on feeder cells obtained from the endometrium of the biological mother (regardless of whether that mother is also the genetic mother). The endometrium is inactivated by H₂O₂, mitomycin C or by irradiation (e.g., gamma radiation). The medium used to support the hESC derivation is serum-free medium comprising KO-DMEM (Richards et al., 2002, Nature Biotechnol 20: 933-936) supplemented with KO-SR (Hovatta et al., 2003, Hum Reprod 18:1404-1409) and bFGF. Outgrowths of embryonic stem cells are monitored daily under phase contrast microscope.

The first passage was undertaken at the moment at which hES cells growing from the ICM filled the whole field of view of a 10× objective, and cells were always passaged at a ratio of 1:2. The ES cell colony should be subdivided into four approximately equal sections. This can be accomplished, for example, using a pasteur pipette with a diameter approximately equal to that of a day 5-day 6 blastocyst to mark a circle to define the outline of the population. A cross is marked in the centre of this circle to divide the colony into four sub-populations. Each sub-population is gently detached, either directly or by moving the plate inside the incubator so that the colonies do not aggregate. Plates are left in the incubator for 48 hours without removing them, followed by continued observation with medium being changed on day 3 or 4.

For the second passage, an additional mechanical dispersion had to be performed by re-plating the newly-formed colonies at a ratio of 1:3. The same treatment as used for the first passage was used again, with dissected colonies being transferred to the new feeder layer with a pasteur pipette. Plates were left to rest in the incubator for 48 hours, and the medium was changed after 3-4 days.

For the third and subsequent passages, enzymatic dispersion was used. Cells were incubated for 6 to 10 minutes with type IV collagenase at 37° C., and/or scraped and then aspirated and resuspended to the appropriate size aliquot (e.g., 30-50 cells per clump) with a pipette. These cells were then transferred to new feeder plates that had previously been prepared.

c. Characterization of embryonic stem cells

Human ES cells will typically have one or more of the following characteristics: a stable karyotype; 23 pairs of chromosomes (including XX or XY); a prolonged ability to divide symmetrically without differentiation; an ability to give rise to differentiated cell types from all three primary germ layers i.e., ectoderm, endoderm and mesoderm; a prolonged telomerase activity; display of non-differentiation markers (e.g., Oct-4, stage-specific embryonic antigens SSEA-3 and SSEA-4, alkaline phosphatase, etc.) which may be detected e.g., by RT-PCR and/or by immunohistochemistry; etc. ES cells of the invention are preferably pluripotent and can be used for potential therapeutic applications in familial subjects.

### REFERENCES

Freed (2002) PNAS USA 99:1755-1757.
Björklund et al. (2002) PNAS USA 99:2344-2349.
U.S. Pat. No. 6,534,052.
Kehat & Gepstein (2003) Heart Fail Rev 8:229-236.
Nir et al. (2003) Cardiovasc Res 58:313-323.
Cheng et al. (2003) Stem Cells 21:131-142.
Richards et al. (2002) Nature Biotechnol 20:933-936.
Hovatta et al. (2003) Hum Reprod 18:1404-1409.
Amit et al. (2003) Biol Reprod 68:2150-2156.
WO03/040346.
Simon et al. (1999) J Clin Endocrinol Metab 84:2638-2646.
Gibco Knockout™ DMEM medium. Invitrogen catalog no. 10829.
Gibco Knockout™ serum replacement. Invitrogen catalog no. 10828.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. All references, patents and patent applications that are recited in this application are incorporated by reference herein in their entirety.

What is claimed is:

1. A method for establishment and use of a human embryonic stem cell line comprising
generating a human embryonic stem cell line;
storing the human embryonic stem cell line in connection with a database for directing use of the human embryonic stem cell line in treating a familial subject.

2. The method of claim 1, wherein generating the human embryonic stem cell line comprises
culturing, on or in the presence of human feeder cells or human serum, a human blastocyst generated using a female donor egg, to generate a human embryonic stem cell line,
wherein the human feeder cells or human serum are derived from a biological or genetic relation of the blastocyst and the feeder cells are mitotically inactivated.

3. The method of claim 1, wherein the human embryonic stem cell line and the database are stored in a human embryonic stem cell line bank.

4. The method of claim 1, wherein the database contains an information record corresponding to the human embryonic stem cell line.

5. The method of claim 3, wherein the database contains an information record corresponding to the human embryonic stem cell line.

6. The method of claim 5, further comprising storing the human feeder cells in the bank and listing the human feeder cells in the information record, wherein the human feeder cells are derived from a biological or genetic relation of the human embryonic stem cell line and were used to generate the human embryonic stem cell line.

7. The method of claim 5, further comprising storing the human serum in the bank and including the human serum in the information record, wherein the human serum is derived from a biological or genetic relation of the human embryonic stem cell line and was used to generate the human embryonic stem cell line.

8-12. (canceled)

13. The method of claim 8, wherein the human feeder cells are bone marrow cells, lung fibroblasts, muscle cells, oral fibroblasts, skin fibroblasts, or tissue-derived stromal cells.

14. The method of claim 1, wherein the database is a computer database.

15. The method of claim 3, wherein the information record comprises a feeder cell sample field or a serum sample field.

16. The method of claim 4, wherein the information record comprises a feeder cell sample field or a serum sample field.

17. The method of claim 3, wherein the information record comprises a stem cell line generation protocol field.

18. The method of claim 4, wherein the information record comprises a stem cell line generation protocol field.

19. The method of claim 3, wherein the information record comprises a stem cell line profile field.

20. The method of claim 4, wherein the information record comprises a stem cell line profile field.

21. The method of claim 3, wherein the information record comprises storage location information and an identification code.

22-24. (canceled)

25. The method of claim 3, further comprising levying a charge for each cryopreserved sample based on length of storage.

26-27. (canceled)

28. The method of claim 1, wherein the human embryonic stem cell line used to treat a familial subject is not screened for pathogen content prior to such use.

29. (canceled)

30. The method of claim 3, wherein the stem cell bank comprises a plurality of samples of human feeder cells.

31. The method of claim 3, wherein the stem cell bank comprises a plurality of samples of human serum.

32. The method of claim 2, wherein the human blastocyst is generated by culturing a zygote in the presence of endometrial epithelial cells.

33. (canceled)

34. The method of claim 1, wherein the human embryonic stem line is generated by the method comprising
culturing, on human feeder cells, a human blastocyst generated using a female donor egg, to generate a human embryonic stem cell line,
wherein the human feeder cells are generic human feeder cells and are mitotically inactivated.

35. A computer readable medium or combination of computer-readable media, containing a program for maintaining a computer-based human embryonic stem cell line database, the program comprising code to effect:
storing information about a human embryonic stem cell line; and
storing an information record in a database.

36-38. (canceled)

39. A method for providing human embryonic stem cell line information to a client, the method comprising computer-implemented steps of:
storing information about a human embryonic stem cell line; and
storing an information record or records that corresponds to the identity and offspring of a donor of the egg and optionally a donor of the sperm in a database.

40. A computer readable medium having computer readable signals stored thereon, the signals defining instructions that, as a result of being executed by a computer, control the computer to perform a process for providing human embryonic stem cell line information, the process comprising acts of:
storing information about a human embryonic stem cell line; and
storing an information record or records that corresponds to the identity and offspring of a donor of the egg and optionally a donor of the sperm in a database.
